(19) United States
(12) Patent Application Publication
(10) Pub. No.: US 2010/0087926 A1
(43) Pub. Date: Apr. 8, 2010

(54) Repair System for Spinal Disc Herniation

(76) Inventors: Michael S. Butler, St. Charles, IL (US); Thomas S. King, Schaumburg, IL (US); Jonathan S. Citow, Glencoe, IL (US)

Correspondence Address: Bruce J. Bowman;Bowman & Associates, 1016 3rd Ave. SW, Suite 106, Carmel, IN, 46032, US

(21) Appl. No.: 12/573,004

(22) Filed: Oct. 2, 2009

Related U.S. Application Data
(60) Provisional application No. 61/102,047, filed on Oct. 2, 2008.

(51) Int. Cl. A61F 2/44; A61B 17/84

(52) U.S. Cl. 623/17.16; 606/300

(57) ABSTRACT

A repair system for a compromised or herniated spine disc is provided. The repair system includes a spine disc patch to cover the herniation, and hooks, fasteners or the like to attach the spine disc patch to the vertebra surrounding the compromised disc. The repair system also includes an application tool to introduce and deploy the spine disc patch and spine disc patch fasteners to and on the disc herniation site. The spine disc patch is composed of a mesh, screen or the like of a biocompatible material with a perimeter, edge or side portion formed of a resilient biocompatible material. The mesh is preferably, but not necessarily, formed of a flexible but non-resilient biocompatible material. The perimeter is preferably, but not necessarily, formed of a flexible, resilient biocompatible material. In this manner, the spine disc patch may fold, bend, crumple or otherwise be deformed for introduction to the disc herniation site within an application tool, then unfold, expand, spread out, unfurl or otherwise open up at and over the disc herniation site when released from the application tool. The application tool consists of an elongate tube connected to a fixed portion of a handle with a rod, disposed for axial movement within the elongate tube. The rod is connected to a pivoting portion of the handle such that the rod axially moves within the elongate tube when the pivoting portion of the handle is moved.

### RELATED APPLICATIONS

This patent application claims the benefit of and/or priority to U.S. Provisional Patent Application Ser. No. 61/102,047 filed Oct. 2, 2008, entitled “Repair System for Spinal Disc Herniation” the entire contents of which is specifically incorporated herein by this reference.

### BACKGROUND OF THE INVENTION

1. Field of the Invention

The present invention relates to systems, devices and/or materials for use in spinal surgery including spinal surgery methods and, more particularly, to systems, devices and/or materials for use in spinal disc surgery.

2. Background Information

Spinal disc herniation is a medical condition affecting the spine in which a tear in the outer, fibrous ring (annulus fibrosis) of an intervertebral disc allows the soft, central portion (nucleus pulposus) to bulge out. This can occur since the disc can be squeezed, stretched and twisted, all in small degrees during various activities. This tear in the disc ring may result in the release of inflammatory chemical mediators which may directly cause severe pain, even in the absence of nerve root compression.

Disc herniation can occur in any disc in the spine, but the two most common places are the cervical portion of the spine (cervical disc herniation) and the lumbar portion of the spine (lumbar disc herniation). The latter is the most common, causing lower back pain (referred to as “lumbago”) and often leg pain as well, in which case it is commonly referred to as sciatica. Lumbar disc herniation occurs more often than cervical disc herniation, and it is one of the most common causes of lower back pain.

Disc herniations can occur from general wear and tear, such as jobs that require constant sitting, but especially jobs that require lifting. Traumatic (quick) injury to lumbar discs commonly occurs from lifting while bent at the waist, rather than lifting while using the legs with a straightened back. Minor back pain and chronic back tiredness is an indicator of general wear and tear that makes one susceptible to herniation on the occurrence of a traumatic event.

Treatment of a herniated or otherwise compromised disc depends on a number of factors including symptoms experienced by the patient, age of the patient, activity level of the patient, and presence of worsening symptoms. Most often, treatments of a herniated disc begin conservatively, and become more aggressive if the symptoms persist. After diagnosing a herniated disc, treatment usually begins with rest and activity modification, ice and heat applications, physical therapy, anti-inflammatory medications, oral steroid medications, other medications and epidural steroid injections. In other cases, surgical treatment of a herniated disc may be recommended.

Currently, the most common herniated disc surgeries are the discectomy, a laminotomy, a laminectomy, and a percutaneous discectomy. A discectomy is the surgical removal of herniated disc material that presses on a nerve root or the spinal cord. The laminotomy and laminectomy are surgeries done to relieve pressure on the spinal cord and/or spinal nerve roots caused by age-related changes in the spine. A laminotomy removes a portion of the thin part of the vertebrae that forms a protective arch over the spinal cord (lamina) while a laminectomy removes all of the lamina on selected vertebrae and also may remove thickened tissue that is narrowing the spinal canal. Either procedure may be done at the same time as a discectomy, or separately. With a percutaneous discectomy, a special tool is inserted through a small incision in the back and disc material is then removed or destroyed to try to reduce pressure on the nerve root.

Given the above, it is desirable to have another system and/or manner of treating and/or repairing spinal disc herniation.

Given the above, it is desirable to have a system and/or manner of supplementing current surgical treatment of spinal disc herniation.

### SUMMARY OF THE INVENTION

The present invention is a repair system, spinal surgical kit and components for a compromised spine disc (i.e. having a problem such as a rupture, tear, crack, fissure, split or other condition (collectively, disc herniation)). The present repair system consists of a spine disc patch to cover the herniation, and hooks, fasteners or the like to attach the present spine disc patch to the vertebra surrounding the compromised disc. The present system also includes application tools or applicators to introduce and deploy the spine disc patch and spine disc patch fasteners to and on the disc herniation site.

In one form, the spine disc patch is composed of a mesh, screen or the like (collectively, mesh) formed of a biocompatible material with a perimeter, edge or side portion (collectively, perimeter) formed of a resilient or elastic biocompatible material. The mesh is preferably, but not necessarily, formed of a flexible (deformable) biocompatible material that allows unfolding thereof. The mesh may be essentially non-resilient or non-elastic. The perimeter is preferably, but not necessarily, formed of a flexible, resilient biocompatible material. In this manner, the spine disc patch may fold, bend, crumple or otherwise be deformed (collectively, fold) for introduction to the disc herniation site in an application tool, then unfold, expand, spread out, unfurl or otherwise open up (collectively, unfold) at and over the disc herniation site when released from the application tool.

The application tool consists of an elongate tube connected to a fixed portion of a handle. A plunger or rod, disposed for axial movement within the elongate tube, is connected to a pivoting portion of the handle such that the rod axially moves within the elongate tube when the pivoting portion of the handle is moved. In this manner, a component disposed in the elongate tube may be deployed therefrom by pivoting movement of the handle. In one form, the axial end of the rod distal the handle is configured with a hook or the like for temporary retention of a folded spine disc patch within the elongate tube and then unfolding deployment therefrom. In another form, the axial end of the rod distal the handle is configured to receive a driving and patch retention end of a spine disc patch hook or fastener for temporary retention of a spine disc patch fastener within the elongate tube and deployment therefrom. In this form, continued movement of the handle causes the end of the rod to axially drive against the driving and patch retention end of the hook to drive and anchor the hook into the vertebra or vertebral bone.

The fasteners or hooks (collectively, fastener(s)) are formed by a flexible or bendable, or semi-flexible or semi-bendable wire or the like having a barb at one end and a driving and patch retention end at the other. In one form, the fasteners have a predefined curve or curvature. In all forms, the fasteners are configured for temporary retention in the application tool then deployment therefrom.

The advantages of this system are increased speed over screws or other attachment devices. Tool sizes may be decreased. Most importantly, in situations where the spinal cord is in close proximity, the present spinal disc repair implant will be low profile so as not to touch the nerve.

### BRIEF DESCRIPTION OF THE DRAWINGS

The above mentioned and other features, advantages and objects of this invention, and the manner of attaining them, will become apparent and the invention itself will be better understood by reference to the following description of embodiments of the invention taken in conjunction with the accompanying drawings, wherein:

FIG. 1 is a top plan view of a spine disc patch fashioned in accordance with the present principles;

FIG. 2 is a side, partial sectional view of an exemplary embodiment of an application tool for the spine disc patch of FIG. 1 with the present spine disc patch in partial deployment from the application tool;

FIG. 3 is an end view of several vertebrae of a spine in which a common spine disc has a rupture, crack, fissure, split or other similar condition (collectively, herniation) that requires attention;

FIG. 3A is a sectional view of the several vertebrae of FIG. 3, taken along line 3A-3A of FIG. 3, particularly showing the herniation of the spine disc needing attention;

FIGS. 4A-C depict the application tool of FIG. 2 being utilized to illustrate the application of a spine disc patch fastener, fashioned in accordance with the present principles, for the spine disc patch of FIG. 1;

FIG. 5 is a top plan view of a treated spine disc of FIG. 3A utilizing the present spine disc patch and spine disc patch fasteners of the present invention;

FIG. 6 is a side view of the treated spine disc of FIG. 5 illustrating the profile of an installed spine disc patch relative to the adjacent vertebral bodies;

FIG. 7 is a perspective view of another exemplary embodiment of an application tool for the present spine disc patch and/or spine disc patch fasteners, wherein several embodiments of spine disc patch fasteners that may be utilized with the present application tool and spine disc patch are shown;

FIG. 8 is a perspective view of a portion of a spine having an existing spine fixation component undergoing spine disc herniation repair utilizing the present spine disc patch and spine disc patch fasteners; and

FIG. 9 is a perspective view of a portion of a spine having an existing spine fixation component undergoing spine disc herniation repair utilizing the present spine disc patch and spine disc patch fasteners.

Like reference numerals indicate the same or similar parts throughout the several figures.

An overview of the features, functions and/or configuration of the components depicted in the various figures will now be presented. It should be appreciated that not all of the features of the components of the figures are necessarily described. Some of these non discussed features as well as discussed features are inherent from the figures. Other non discussed features may be inherent in component geometry and/or configuration.

### DETAILED DESCRIPTION OF EMBODIMENTS OF THE INVENTION

The invention is a repair system for a compromised spine disc (i.e. a disc having a problem such as a rupture, tear, crack, fissure, split, herniation or other condition. The present repair system consists of a spine disc patch to cover, extend about or over the tear, and hooks or fasteners to attach or fasten the spine disc patch to the vertebrae surrounding the compromised disc. The present repair system also includes tools to aid in the insertion, introduction, placement and/or deployment of each component.

Referring to FIG. 1, there is shown an exemplary embodiment of a spine disc patch, generally designated 10, fashioned in accordance with the principles of the present invention. The spine disc patch 10 is formed of a mesh, screen, screen mesh or the like 12 of a biocompatible material. The mesh 12 is preferably flexible to allow the mesh 12 to be folded, bent, crumpled or otherwise deformed (collectively, fold) for introduction to a disc herniation/repair site in an application tool, and then be unfolded, expanded, spread out, unfurled or otherwise opened up (collectively, unfolded) at and over the disc herniation/repair site when released from the application tool. The spine disc patch 10 also includes a perimeter, edge or side portion 14 (collectively, perimeter) formed of a resilient or elastic biocompatible material. The perimeter is preferably, but not necessarily, formed of a flexible, resilient biocompatible material. In this manner, the spine disc patch may fold, bend, crumple or otherwise be deformed (collectively, fold) for introduction to the disc herniation site in an application tool, then unfold, expand, spread out, unfurl or otherwise open up (collectively, unfold) at and over the disc herniation site when released from the application tool.

Preferably, but not necessarily, the spine disc patch 10 has corner apertures 15 for receipt of a fastener (as described below). Additionally, the spine disc patch 10 may include a center aperture 16.

The mesh 12 is fine enough to prevent the mucoprotein gel from passing through and effectively patch the tear. The perimeter 14 of the mesh 12 will be stiffer or resilient such that the patch 10 is fully expanded in its relaxed state. This will allow the patch 10 to be folded into the insertion tool so it may be passed through a small incision and directed to the tear. Once released from the tool, it will deploy to full size to cover the tear.

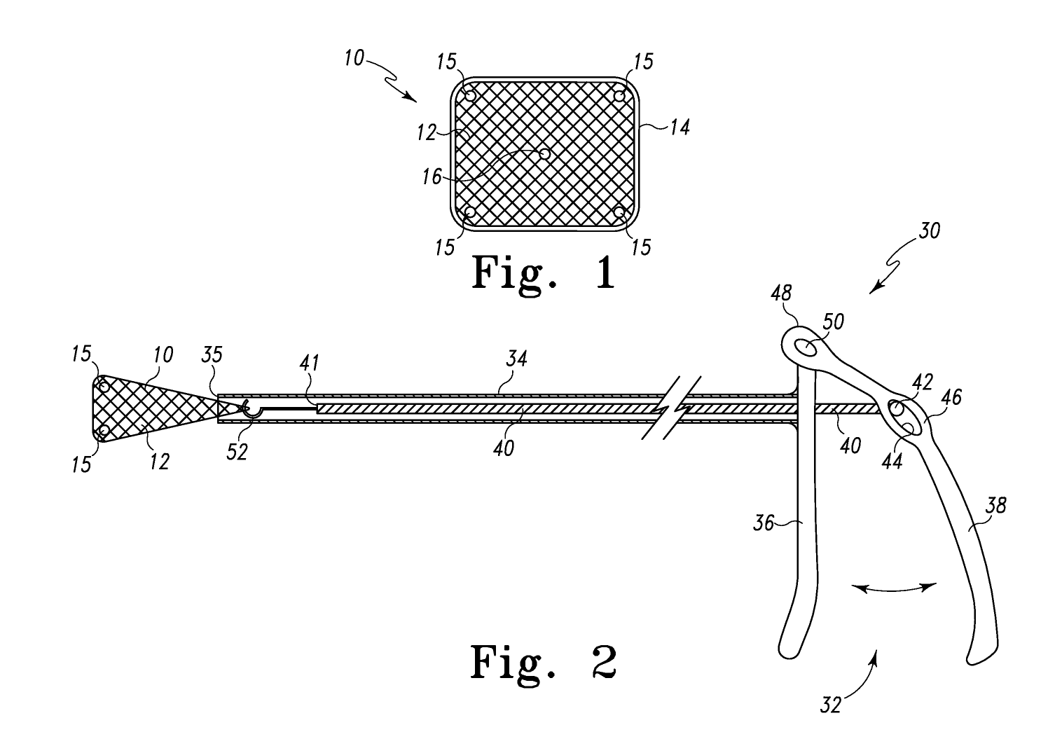

FIG. 2 depicts an application tool 30 for introducing the patch 10 to a disc repair site and deploying the patch 10 onto and/or over the disc repair site. The application tool 30 includes a handle 32 and an elongate (hollow) tube 34. The elongate tube 34 is attached to a stationary portion 36 of the handle 36. A movable portion 38 of the handle 38 is pivotally connected to the stationary portion 36. Particularly, the moving portion 38 has a boss 48 on an upper end thereof that connects to pivot 50 of the stationary handle 36. The movable portion 38 thus pivots about the stationary portion 36 as represented by the double-headed arrow. The moving portion 38 includes a boss 46 that defines an opening 44 the purpose of which is described below.

A rod or plunger 40 is axially movably disposed in the elongate tube 34 and extends through the stationary portion 36 of the movable handle 32. An end 42 of the rod 40 is retained in the opening 44 of the boss 46 of the movable portion 38 of the handle 32. Movement of the handle portion 38 axially moves the rod 40 within the elongate tube 34 as represented by the double-headed arrow. This axial movement of the rod 40 allows the retention of the spine disc patch 10 to be stored within the tube 34 and then deployed therefrom. In FIG. 2, the spine disc patch 10 is shown being deployed from the applicator 30 (and particularly from the end 35 of the tube 24. In this embodiment, the applicator 30 is installed with a spine disc patch holder 50 here shown as a hook. The hook 50 is attached to end 41 of the rod 40 such that axial movement of the rod 40 causes like axial movement of the hook 50. The spine disc patch 10 is held by the hook 50 for deployment.

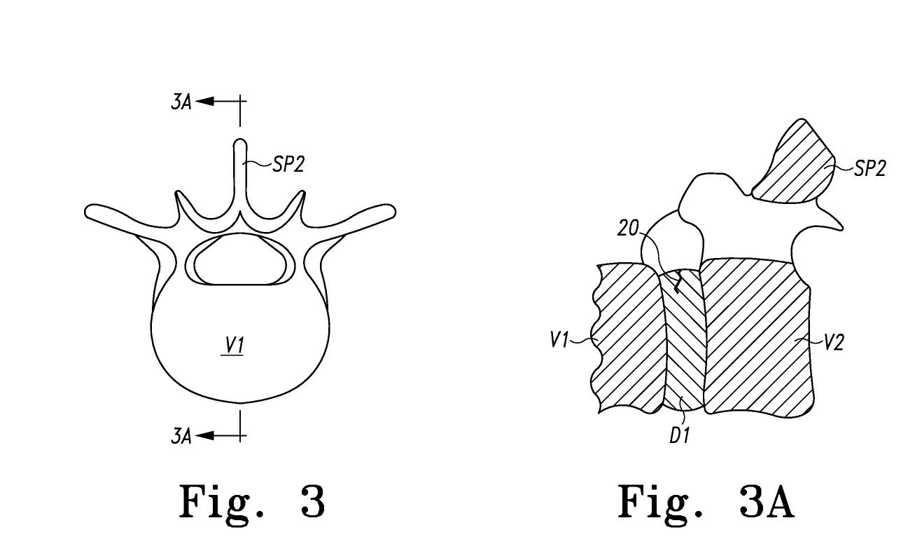

FIGS. 3 and 3A depict a portion of a spine and particularly first and second, adjacent vertebrae V1 and V2 with a compromised spine disc D1 situated between the vertebrae V1, V2. The compromised spine disc D1 has a tear 20 therein which is in need of repair.

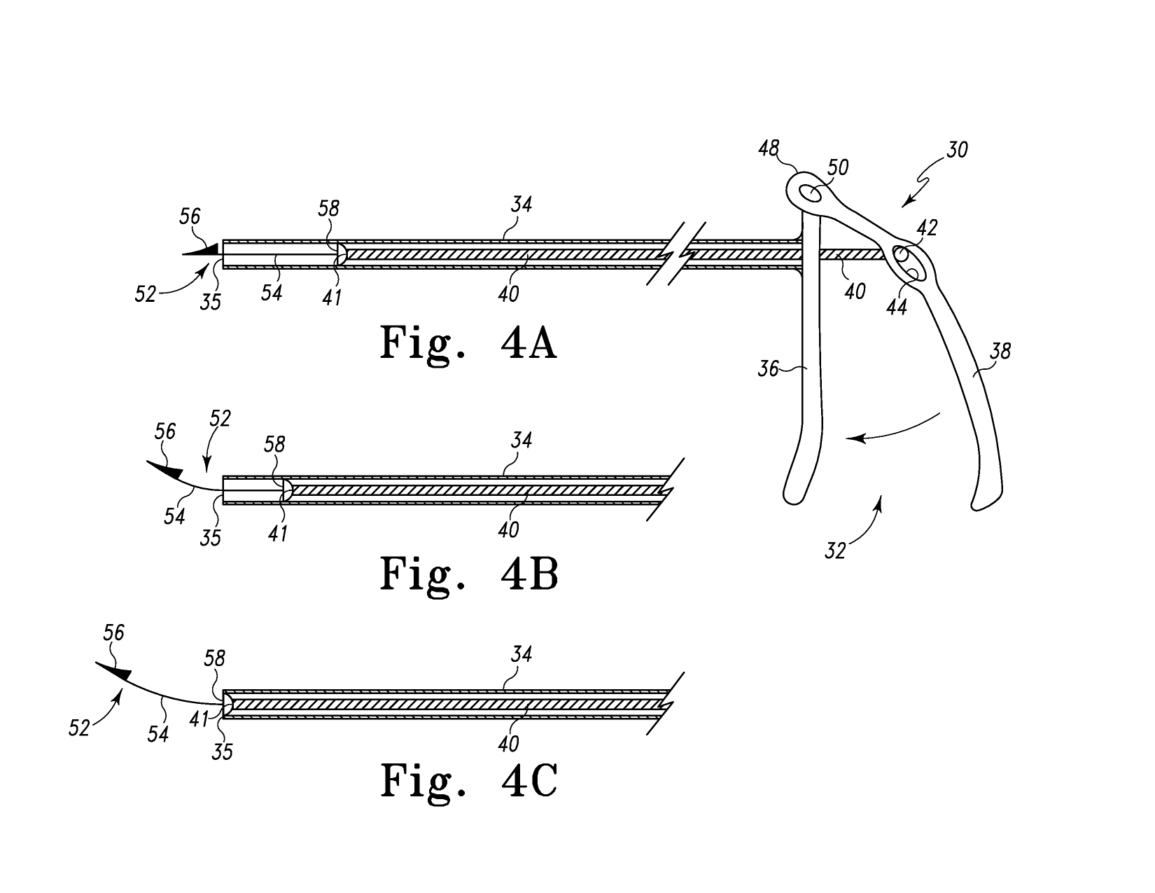

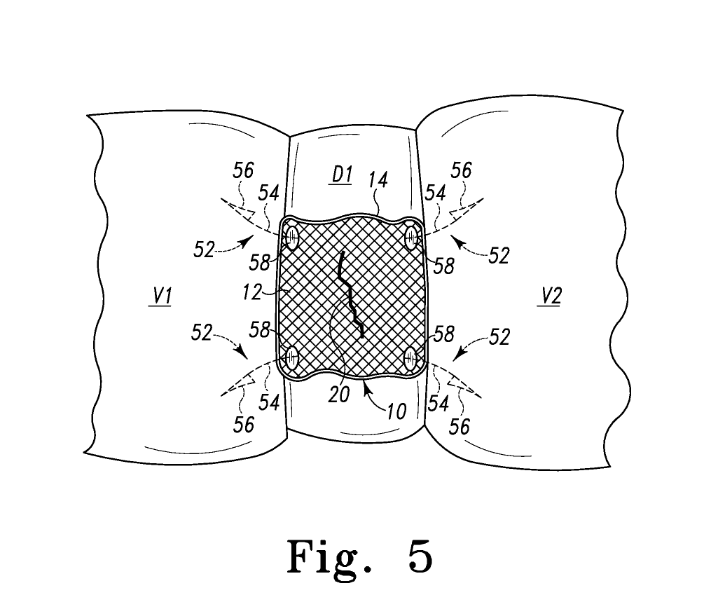

FIGS. 4A through 4C depict a manner of utilizing the applicator 30 for deploying spine disc patch hooks or fasteners to attach the spine disc patch 10 onto the disc repair site, and particularly to the vertebrae or vertebral bone adjacent the compromised spine disc D1. In this use/embodiment, the applicator 30 does not have a hook at the end of the rod 40. Instead, the end 41 of the rod 40 is flat and/or configured to receive and/or abut a head or head end 58 of a spine disc patch fastener 52.

The fastener 52 includes a flexible, curved member or wire 54 having a barb or barbed end 56 distal the head 58. The barb is configured for permanent placement of the fastener 52 into the vertebral bone to hold the spine disc patch 10 onto and/or over the disc compromise, tear or repair site. FIGS. 4A through 4C illustrate the progression of the fastener 52 during deployment. While in the applicator tool, the fastener 52 is held straight against its natural curvature or curve bias to aid in placement. The barb 56 is pushed through the mesh 12 (or into the apertures 15) and into the vertebral bone. Once started, the tool will continue to push against the head 58 of the fastener to push the fastener out of the tube 34, whereupon the fastener curves upon exit. It should be appreciated that other styles of fasteners (e.g. straight fasteners) may be used. When the head 58 is flush to the mesh 12 and the spine disc patch 10 to the vertebral bone, the fastener 52 is installed. Using a curved shape increases pullout resistance, and the head 58 holds the spine disc patch 10 in place. Four fasteners are typically used in the same application.

The advantages of this system are increased speed over screws, anchoring mechanisms, anchoring site, control of anchor placement and the reduction of unnecessary instrumentation that may be implanted to accomplish similar outcomes. Tool sizes may be decreased.

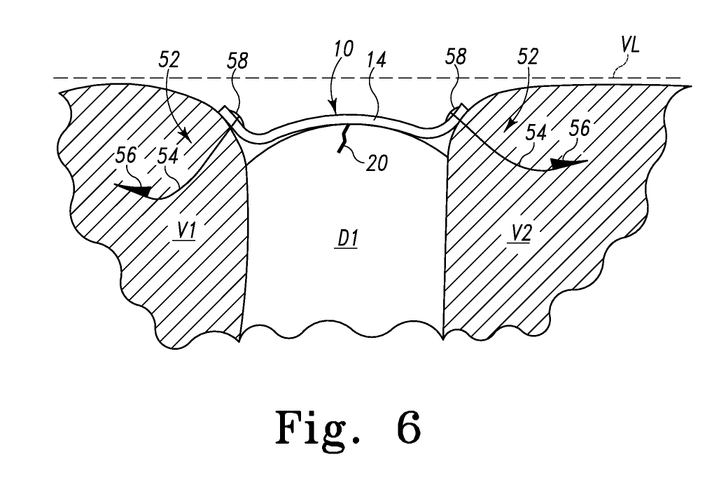

Referring to FIGS. 5 and 6, there is shown the compromised disc D1 having undergone a disc repair in accordance with the present principles. The spine disc patch 10 has been deployed over the disc repair site and anchored, fastened or attached to the vertebral bone (i.e. vertebrae V1 and V2). It can be seen that the implant can be low profile so as not to touch the nerves (see e.g., FIG. 6). This is especially good for cases where the spinal cord is in close proximity to the repair site/spine disc patch.

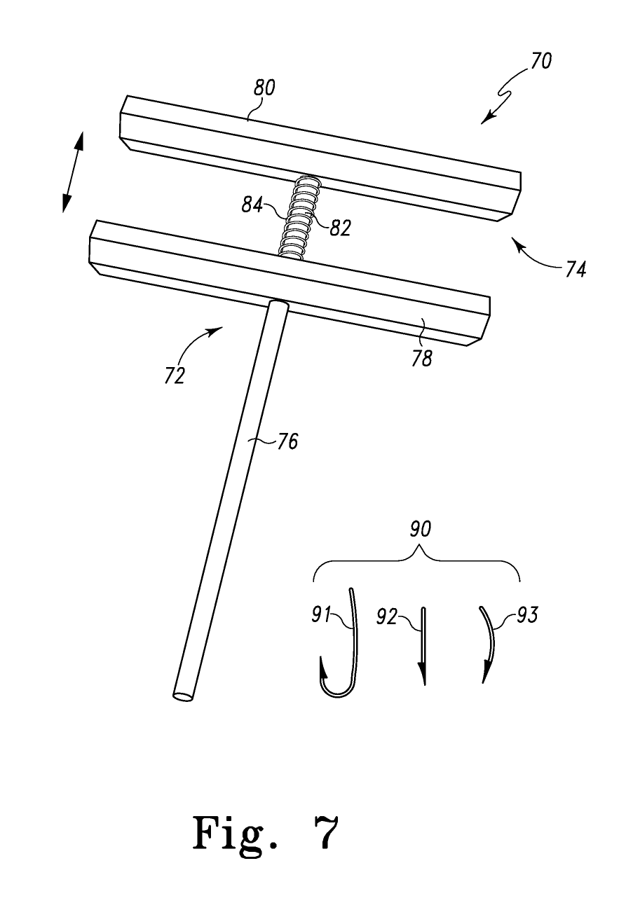

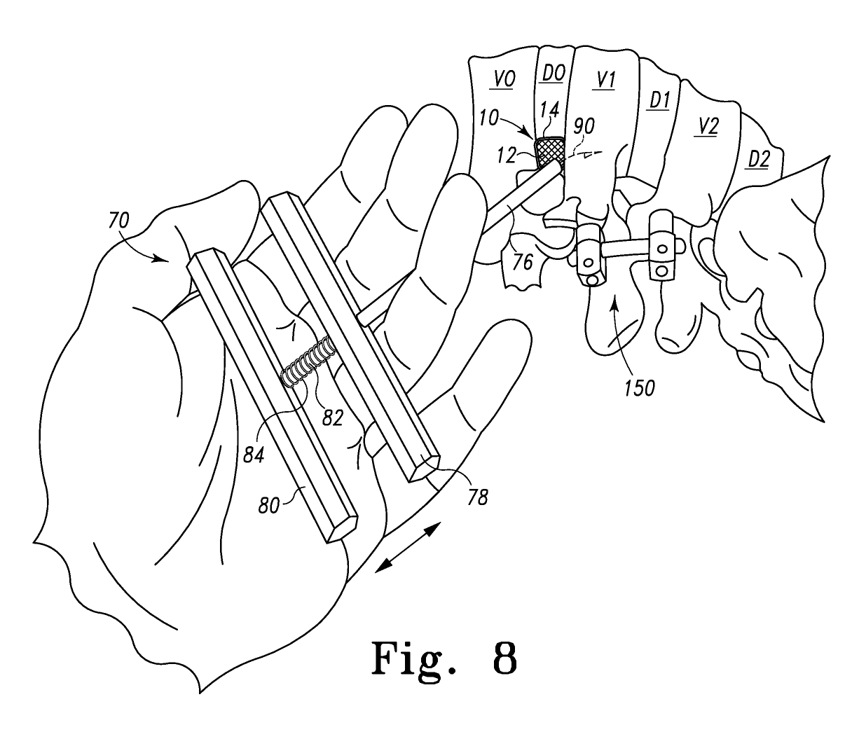

Referring to FIG. 7 shows an exemplary embodiment of a hook/fastener application or installation tool (applicator or installer) generally designated 70 and various exemplary hooks/fasteners generally designated 90. Various exemplary hooks/fasteners 91, 92 and 93 are shown that can be used either with the applicator 70 or the applicator 30. Likewise, the applicator 70 may be used to deploy the spine disc patch 10. The applicator 70 is characterized by a first part 72 and a second part 74 that is movable relative to the first part 72 (as represented by the double-headed arrow). It should be appreciated that the nomenclature first and second is arbitrary.

The first part 72 has a hollow tube or cylinder 76 with a handle 78 situated at one end thereof and transverse to the tube 76. The tube 76 is hollow and is open at both the handle end and the free end thereof. The second part 74 has a handle 80 to which a rod 82 transversely extends. The rod 82 is sized to be received in the tube 76. A spring 84 is situated on the rod 82 between the handle 78 of the first part 72 and the handle 80 of the second part 74. The spring 84 provides a biasing of the second part 74 away from the first part 72. This naturally draws a hook/fastener 90 into the tube 76. A positive force applied against the handle 80 relative to the handle 78 installs a fastener/hook.

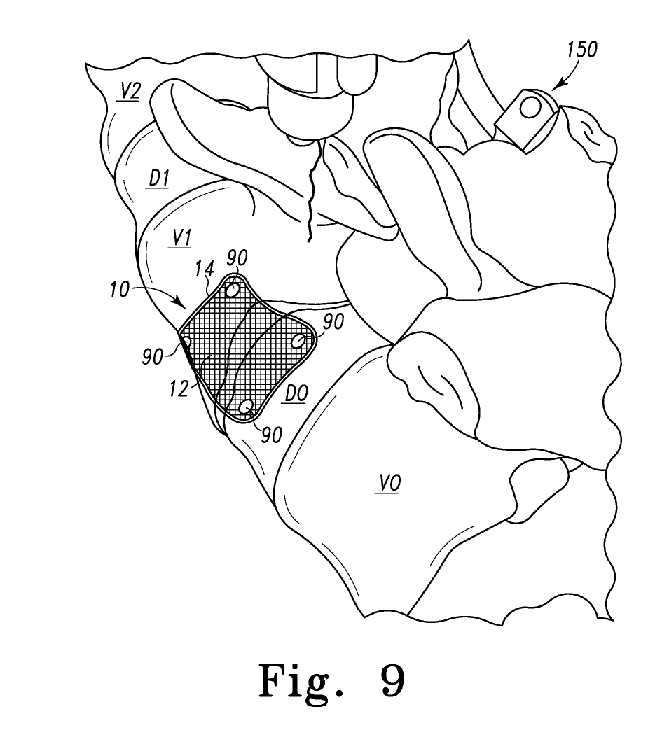

This can be seen in FIG. 8. FIG. 8 shows the installation of a fastener 90 into the spine disc patch 10 and the vertebra V1 via the installation tool 70. Other fasteners 90 will be used to attach the spine disc patch 10 onto and/or over the disc repair site. In FIG. 9, a spine disc patch 10 has been fully attached over and/or onto the repair site. Four fasteners 90 have been used.

While the invention has been illustrated and described in detail in the drawings and foregoing description, the same is to be considered as illustrative and not restrictive in character, it being understood that only preferred embodiments have been shown and described and that all changes and modifications that come within the spirit of the invention are desired to be protected.

What is claimed is:

1. A spinal implant comprising:
a body of a biocompatible mesh; and
an edge situated about the perimeter of the body and formed of an elastic biocompatible material.

2. The spinal implant of claim 1, wherein the body is rectangular.

3. The spinal implant of claim 1, further comprising:
a plurality of apertures formed in the body.

4. The spinal implant of claim 3, wherein an aperture of the plurality of apertures is situated at ends of the body.

5. The spinal implant of claim 1, wherein the body is formed of a flexible biocompatible material.

6. The spinal implant of claim 5, wherein the flexible biocompatible material of the body is non-elastic.

7. A spinal implant for the repair of a compromised spine disc comprising:
a spine disc patch formed of a biocompatible mesh with an elastic perimeter; and
a plurality of spine disc patch fasteners configured to extend through the spine disc patch, extend into vertebral bone adjacent the compromised spine disc, and hold the spine disc patch onto the compromised spine disc.

8. The spinal implant of claim 7, wherein the spine disc patch is rectangular.

9. The spinal implant of claim 7, further comprising:
a plurality of apertures formed in the spine disc patch.

10. The spinal implant of claim 9, wherein an aperture of the plurality of apertures is situated at ends of the spine disc patch.

11. The spinal implant of claim 7, wherein the biocompatible mesh is formed of a flexible biocompatible material.

12. The spinal implant of claim 11, wherein the flexible biocompatible material of the mesh is non-elastic.

13. The spinal implant of claim 7, wherein each spine disc patch fastener is formed as a flexible shaft having a barb at one end and a head at another end.

14. The spinal implant of claim 13, wherein each spine disc fastener has an innate curvature.

15. The spinal implant of claim 13, wherein the flexible shaft is formed of a biocompatible wire.

16. A surgical kit for repair of a herniated spine disc, the surgical kit comprising:
a body formed of a biocompatible mesh with a biocompatible elastic perimeter; and
a plurality of biocompatible fasteners configured to extend through the body, extend into vertebral bone adjacent the compromised spine disc, and hold the body onto the compromised spine disc.

17. The surgical kit of claim 16, wherein each fastener is formed as a flexible shaft having a barb at one end and a head at another end.

18. The surgical kit of claim 17, wherein each fastener has an innate curvature.

19. The surgical kit of claim 17, wherein the flexible shaft is formed of a biocompatible wire.

20. The surgical kit of claim 16, wherein the mesh of the body is formed of a flexible, non-elastic biocompatible material.
